(19) United States
(12) Patent Application Publication
(10) Pub. No.: US 2015/0231355 A1
(43) Pub. Date: Aug. 20, 2015

(54) Support Apparatus for Front-Mounted Breathing Tube

(71) Applicant: Shine Ball Enterprise Co., Ltd., Chiayi County (TW)

(72) Inventor: Yen-Ting Kuo, Chiayi County (TW)

(73) Assignee: Shine Ball Enterprise Co., Ltd., Chiayi County (TW)

(21) Appl. No.: 14/184,770

(22) Filed: Feb. 20, 2014

(51) Int. Cl. A61M 16/06; A61M 16/08; A61M 16/04

(52) U.S. Cl. CPC A61M 16/0638; A61M 16/0497; A61M 16/0493; A61M 16/0683; A61M 16/0875

(57) ABSTRACT

The invention relates to a support apparatus for a front-mounted breathing tube, comprising a breathing tube inserted into a breathing tube positioning member, two ball-pivot portions arranged symmetrically on one side thereof, and a concave slot is formed between the two ball-pivot portions for swinging elastically; two forehead positioning members are arranged symmetrically and a head-band connecting portion is at the outer side thereof; the inner side includes a connecting portion having ball-socket portions for receiving the ball-pivot portions therein. With the ball-pivot portions on the breathing tube positioning member and with the ball-socket portions of the forehead positioning members, the forehead poisoning members can swing in different directions and angles. Therefore, the breathing tube is held by the user properly and the forehead positioning members is fitted automatically onto the forehead thereof with perceptions according to the forehead size, shape and curvature and with wearing comforts for the user.

### BACKGROUND OF THE INVENTION

1. Technical Field

The present invention relates to a support apparatus with a front-mount breathing tube fixed to the forehead of a user, such that an apparatus capable of adjustment of directions and angles is formed by the connecting head-band between the two forehead positioning members and the breathing tube positioning member of the support apparatus in order for people with different head sizes and forehead shapes to wear the apparatus well and comfortably.

2. Description of Related Art

The mouthpiece design of a general front-mounted breathing tube is different from the breathing tube for snorkeling, in which the mouthpiece of the front-mounted breathing tube is designed to be at the front for a user wearing the head-band to inhale and exhale stably. In addition, to allow the breathing tube to be worn on the head of the user stably without moving and rotating left to right, a support is typically worn on the forehead of the user via a head-band such that the breathing tube can be fitted onto a breathing tube fitting unit provided at the front end of the support.

For example, patent No. M421123 published in Taiwan discloses that a breathing tube positioning member is provided between two forehead positioning members symmetrically left and right and is fastened with a screw to form a ring shape in order for the breathing tube to fit through tightly. In addition, a head-band is connected between the outer sides of the two forehead positioning members, so that once the head-band is tightened up, the two forehead positioning members can be fitted onto the forehead of the user closely, such that the breathing tube is prevented from swinging or wobbling left and right at the front of the forehead of the user. Furthermore, according to the forehead protrusion level of users, with a rotatable design of the breathing tube positioning member, the breathing tube can be adjusted to swing at different angles back and forth on the forehead in order for the user to hold the mouthpiece of another end of the breathing tube in his or her mouth properly and comfortably.

However, in order to allow the two forehead positioning members to be attached to the forehead of the user firmly, the head-band must be tightened up such that it is bent in shape. However, in case that there is a reinforcement rib provided on the forehead positioning member, the bending capability of the forehead positioning members would be affected. Therefore, in order for the two forehead poisoning members to fit onto the forehead of the user firmly, there is a need to increase the fitting force on the forehead, which results in an overly strong force on the head-band and causes the user to feel uncomfortable with respect to the head or even feel headache due to such overly fitting force on the head.

### BRIEF SUMMARY OF THE INVENTION

In view of the above, to improve the aforementioned two forehead positioning members of the known support apparatus for a front-mounted breathing tube and to overcome the problem of the over tightening of the head-band due to different shapes of foreheads of users, the present invention provides a solution in which a connection portion between the opposite inner sides of the two forehead positioning members includes a ball-socket portion respectively in the interior thereof for the two symmetrical ball-pivot portions to be arranged on the breathing tube positioning member of the breathing tube passing therethrough to be firmly fitted and received therein, while a concave slot is formed between the two ball-pivot portions in order for the two ball-pivot portions to bend and swing back and forth; furthermore, the two forehead positioning members are allowed to swing and can be fitted onto foreheads of different shapes of users without the condition of over tightening of the head-band.

The technical solution adopted in the present invention to overcome the technical problem is that: the present invention comprises two forehead positioning members arranged symmetrically left and right, and a breathing tube positioning member connected between the two forehead positioning members and provided to allow the breathing tube to be inserted and positioned therein; the outer side of each forehead positioning member includes a head-band connecting portion and a connecting portion having a ball-socket portion in the internal thereof is provided in the inner side; the breathing tube positioning member is provided for the breathing tube to be firmly inserted therein and includes ball-pivot portions arranged symmetrically left and right on one side thereof and used to be fitted and received firmly inside the aforementioned ball-socket portions; thereby the two forehead positioning members are allowed to swing in different directions and angles with the attachment between the aforementioned ball-pivot portions and the ball-socket portions.

According to the aforementioned solution, a concave slot is formed between the two ball-pivot portions and provided for the ball-pivot portions at each side to elastically swing back and forth.

According to the aforementioned solution, one side of the connecting portion of the forehead positioning member is provided with a cut-out slot cut to a center portion of the ball-socket portion.

Therefore, by using the left and right ball-socket portions arranged on the two forehead positioning members of the present invention to contain firmly the two ball-pivot portions arranged on the breathing tube positioning member therein, the two forehead positioning members are able to rotate freely and to swing back and forth in or order to accommodate different head sizes and curvature of the shape of the foreheads of users while allowing the two forehead positioning members to be worn firmly and comfortably on the forehead of the user as well as allowing the mouthpiece to be held properly by the user in his or her mouth.

### BRIEF DESCRIPTION OF THE SEVERAL VIEWS OF THE DRAWINGS

FIG. 1 is a perspective view showing the assembly of the present invention.

FIG. 2 is an exploded view of the present invention.

FIG. 3 is an illustration viewed from another direction of the breathing tube positioning member of the present invention.

FIG. 4 is a side view showing the actuation of the breathing tube of the present invention.

FIG. 5 is a top view of the present invention.

FIG. 6 is an illustration showing the forehead positioning member of the present invention as it swings outward.

FIG. 7 is an illustration showing the forehead positioning member of the present invention as it swings inward.

### DETAILED DESCRIPTION OF THE INVENTION

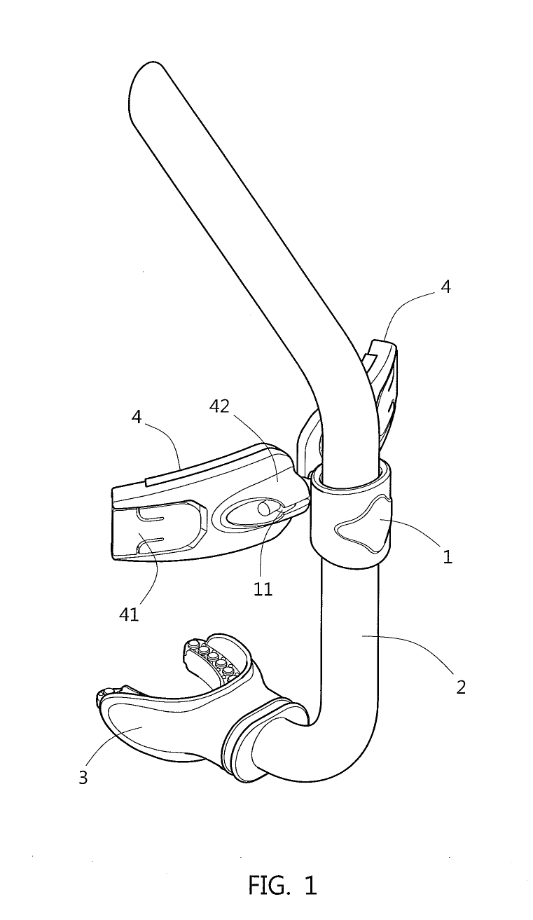

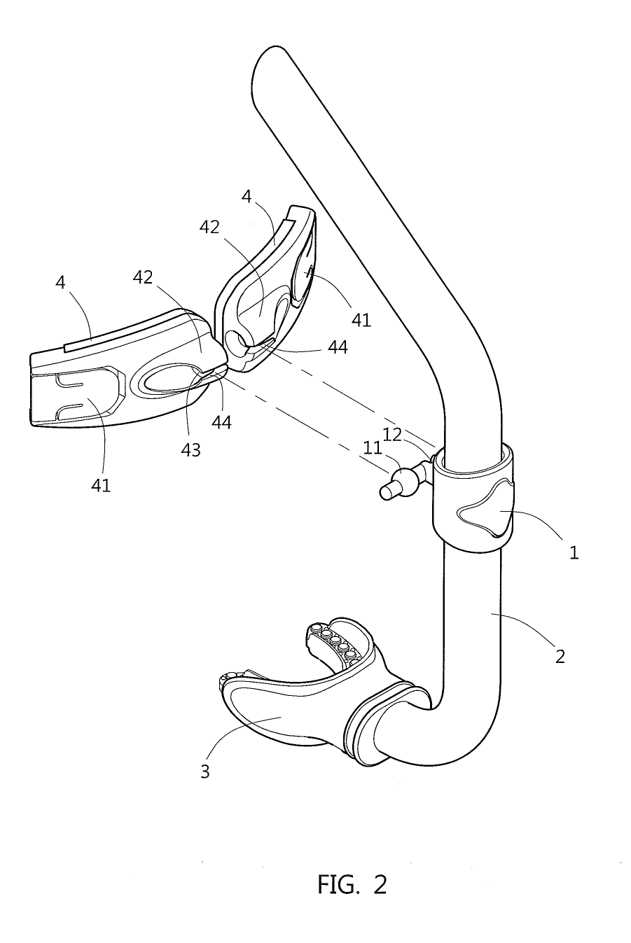

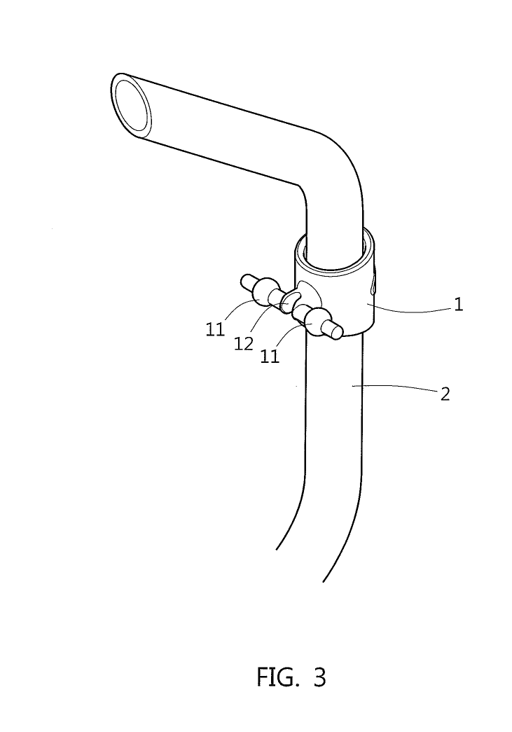

As shown in FIG. 1, FIG. 2 and FIG. 3, the present invention comprises a breathing tube 2 having a mouthpiece 3 attached to a lower end thereof and inserted into a breathing tube positioning member 1. One side of the breathing tube positioning member 1 is provided with two opposite ball-pivot portions 11 and a concave slot 12 is formed between the two ball-pivot portions 11 such that the ball-pivot portions 11 on the two sides are able to use the concave slot 12 as a supporting point to elastically swing back and forth.

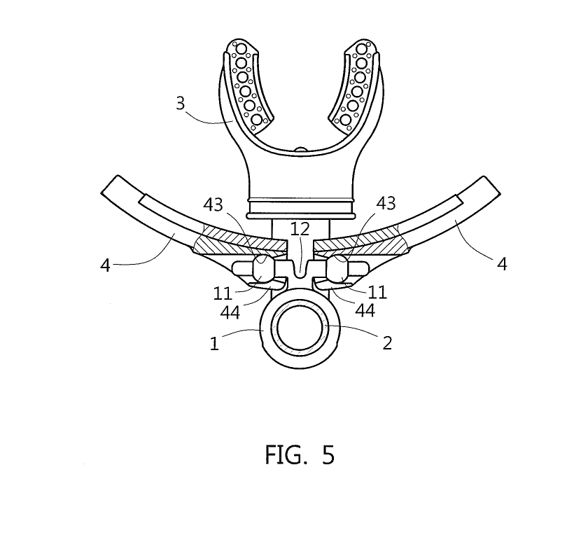

In addition, the present invention comprises two forehead positioning members 4 arranged symmetrically left and right; also, each outer side thereof is provided with a head-band connecting portion 41 for connecting to a head band, and each inner side is provided with a connecting portion 42 having a ball-socket portion 43 formed in the interior thereof; furthermore, an outer side of the connecting portion 42 is provided with a cut-out slot 44 cut to the center of the ball-socket portion 43. Therefore, as shown in FIG. 5, the two ball-socket portions 11 of the breathing tube positioning member 1 are fitted and received firmly inside the left and right ball-socket portions 43 of the forehead positioning members 4, respectively.

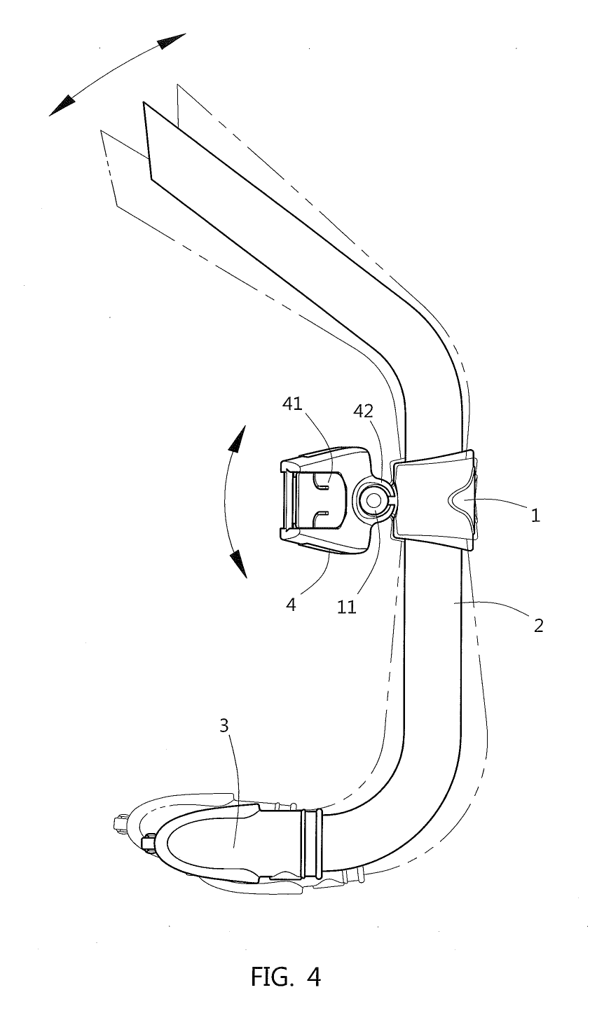

Accordingly, as shown in FIG. 4, with the attachment between the ball-pivot portions 11 arranged on the aforementioned breathing positioning tube 1 and the left and right ball-socket portions 43 arranged on the two forehead positioning members 4, the breathing tube 2 is able to swing back and forth for adjustments in accordance with different curvature of the shapes of the foreheads of different users in order for the user to hold the mouthpiece 3 in his or her mouth properly.

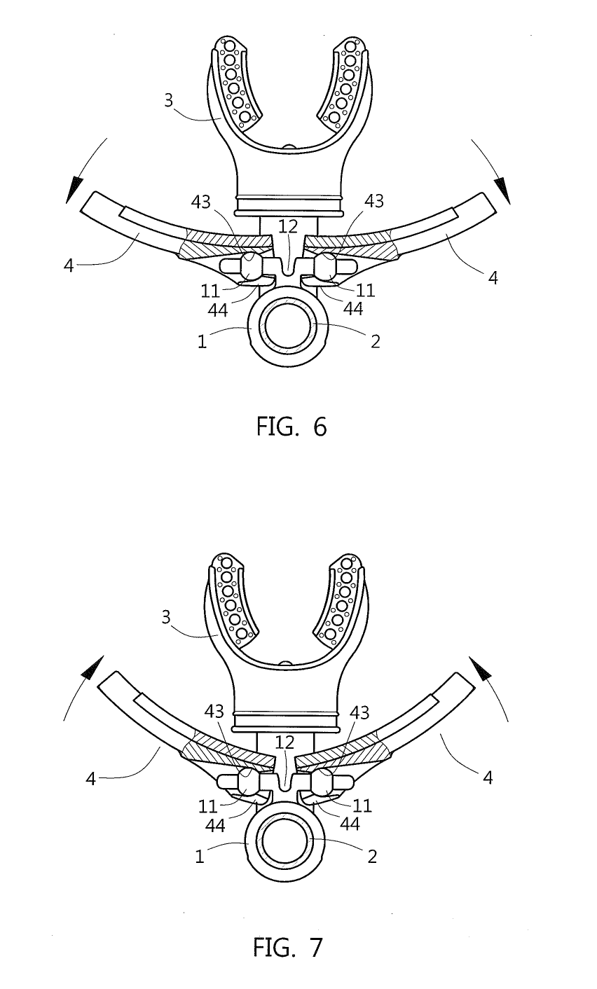

Furthermore, as shown in FIG. 6 and FIG. 7, with the attachment between the ball-pivot portions 11 arranged on the aforementioned breathing positioning tube 1 and the left and right ball-socket portions 43 arranged on the two forehead positioning members 4, the two forehead positioning members 4 are able to swing for adjustments in different directions and angles in accordance with the head sizes and curvature of the shape of the foreheads of different users such that not only the two forehead poisoning members 4 can be fitted onto the forehead of the user completely and firmly, but also the problem associated with the over tightening of the head-band due to the adjustment of the fitting of the forehead positioning members 4 on the forehead can be overcome. In other words, the strength of the tightening force of the head-band can be maintained in a proper range in spite of the difference in the size of the head shape or the curvature of the forehead of the user during the adjustment of the head-band when wearing the front-mounted breathing tube; therefore, the unfavorable occurrence of over tightening thereof can be prevented.

What is claimed is:

1. A support apparatus for a front-mounted breathing tube, comprising two forehead positioning members arranged symmetrically left and right, and a breathing tube positioning member connected between the two forehead positioning members and provided for the breathing tube to be inserted and positioned therein, wherein:
the two forehead positioning members include a head-band connecting portion on each outer side thereof, respectively, and each inner side thereof is provided with a connecting portion having a ball-socket portion arranged at an interior thereof;
the breathing tube positioning member is provided to allow the breathing tube to be inserted therein firmly and includes ball-pivot portions arranged symmetrically left and right on one side thereof and provided to be fitted and received firmly inside the aforementioned ball-socket portions;
thereby the two forehead positioning members are allowed to swing in different directions and angles with the combination of the aforementioned ball-pivot portions and the ball-socket portions.

2. The support apparatus for a front-mounted breathing tube according to claim 1, wherein a concave slot is formed between the two ball-pivot portions and provided for the ball-pivot portion on each side to elastically swing back and forth.

3. The support apparatus for a front-mounted breathing tube according to claim 1, wherein one side of the connecting portion of the forehead positioning member is provided with a cut-out slot cut to a center portion of the ball-socket portion.
